(19) United States
(12) Patent Application Publication
(10) Pub. No.: US 2026/0354796 A1
(43) Pub. Date: Nov. 12, 2026

(54) MEDICAL ENDOSCOPE WITH SHEATH

(71) Applicant: AXESS VISION TECHNOLOGY, JOUE LES TOURS (FR)

(72) Inventors: Xavier DIEUDONNÉ, MONTS (FR); Emmanuel HALLAUER, SACHE (FR)

(73) Assignee: AXESS VISION TECHNOLOGY, JOUE LES TOURS (FR)

(21) Appl. No.: 19/153,528

(22) PCT Filed: Feb. 5, 2024
(86) PCT No.: PCT/FR2024/050144

§ 371 Date: Aug. 4, 2025

(30) Foreign Application Priority Data
Feb. 6, 2023 (FR) FR2301089

(51) Int. Cl. A61B 8/00; A61B 8/12; A61M 25/00; A61M 25/06

(52) U.S. Cl. CPC A61B 8/445; A61B 8/12; A61B 8/4416; A61M 25/0009; A61M 25/0668; A61M 2025/0681

(57) ABSTRACT

The invention relates to a medical endoscope including an insertion tube entirely covered with at least one biocompatible, sealed sheath, comprising a sleeve and having a distal end transparent to the ultrasound and light waves, the sleeve being flexible, stretchable, matching the shape of the insertion tube, having a hardness ranging from 1 to 70 Shore D and a thickness of 10 to 300 μm and having a tearing system. The invention also relates to a method for preparing a medical endoscope according to the invention. Finally, the invention relates to a method for preparing a medical endoscope for reuse.

### TECHNICAL FIELD

The invention relates to the technical field of medical endoscopes.

The medical endoscopes in accordance with the invention allow access to the inner surface of a hollow organ, a cavity or a natural or artificial duct of the human body in order to perform various operations for therapeutic, surgical or diagnostic purposes. The medical endoscopes in accordance with the invention are used for the inspection of all the inner parts of the human body accessible by the natural or artificial routes. For example, the medical endoscopes according to the invention can be used in the field of the urinary tract, the gastrointestinal tract, the respiratory system, the cardiovascular system, the trachea, the sinus cavity, the female reproductive system, the abdominal cavity or any other part of the human body to be explored by a natural or artificial route.

### PRIOR ART

Generally, a medical endoscope includes, as described for example by patent application WO 2014/106510, a control handle to which an insertion tube is fixed. This tube includes a distal head equipped with an optical visualization system for illuminating and examining the organ, the cavity or the duct of the human body. Upstream of this distal head, the insertion tube includes a bending structure or a tip deflection portion for orienting the distal head using one or more actuation cables mounted inside the insertion tube.

During its use by the practitioner, the insertion tube is inserted into the inspected natural cavity or duct. After use, the medical endoscope, and particularly the insertion tube, is either discarded, cleaned and disinfected or even sterilized, for reuse.

Various means can be used to clean and disinfect the insertion tube. However, disinfection may not be sufficiently effective and may constitute a source of nosocomial infection for the patient. Also, alternative methods have been developed. Particularly, the use of a sheath or a sock wrapping all or part of the insertion tube of the medical endoscope during its use has been described.

Particularly, document GB 2 336 540 describes a flexible sheath into which an endoscope can be inserted for use by a practitioner. The distal end of the sheath includes a transparent window to ensure good visibility. The proximal end includes a seal to prevent any contamination. The sheath is removed after use by pulling on the proximal end, which is not easy for the practitioner and carries risks of deterioration of the medical endoscope. In addition, this sheath requires precise adjustment when it is put in place, in order to properly adjust the position of the transparent window, which may be incompatible with life-threatening emergency situations due to the time it takes to put the sheath in place on the endoscope.

U.S. Pat. No. 5,827,177 describes the use of a sheath to protect the endoscope from any contamination during an endoscopy. This sheath includes a tip at its distal end, and an insulating sheet on the inside for limiting the friction of the actuation cables and the insertion tube in particular, and to ensure that they are held in position.

U.S. Pat. No. 8,360,968 relates to a protective sheath to isolate an endoscope from the tissues during use, and thus avoid any cleaning or sterilization between the patients. This sheath comprises a window at its distal end, as well as at least one light source mounted therein. Again, this sheath requires precise adjustment when it is put in place, in order to properly adjust the position of the transparent window, which may be incompatible with life-threatening emergency situations due to the time it takes to put the sheath in place on the endoscope.

Document EP 0 520 743 describes an endoscope provided with a sheath comprising a tubular sleeve and a lens at one end of the sleeve, fixed together by heating or by means of a solvent or an adhesive. According to one particular embodiment, the sheath is flexible. The sheath does not comprise a tearing system and is then removed like a stocking taken off a person's foot, which is not easy for the practitioner and carries increased risks of deterioration and contamination of the medical endoscope. In addition, this sheath requires not only precise adjustment when it is put in place, but also drying or cooling depending on the fixation technique used. Also, putting the sheath in place and assembling it is long and requires specific equipment.

These sheaths described in the prior art therefore have the drawback of not being able to be easily taken off after use of the endoscope, and in view of its reuse by the practitioner.

Moreover, document US 2012/0010468 describes the use of a sheath covering an insertion tube of a medical endoscope, to prevent any contamination between two uses of the endoscope. The sheath is tubular in shape, has an orifice at each of its ends and includes a flap with an adhesive shape to close it. This sheath is torn to allow the use of the endoscope. This sheath is not suitable for use during the endoscopy due to the thickness of the sheath, its rigidity and the orifices which do not guarantee the absence of contamination by the tissues.

Document US 2004/210110 describes a medical endoscope provided with a sheath comprising a disposable tubular sleeve and a cap. The sleeve comprises adhesive areas, as well as a wire for tearing it, preferably disposed longitudinally from one end of the sleeve to the other.

Document WO 97/29679 describes an endoscope comprising a thick sheath for isolating the insertion tube from the body of the examined patient, and which can be taken off after use of the endoscope by means of a tearing cord. The sheath comprises a first longitudinal channel for the passage of the flexible tube of the endoscope, and a series of longitudinal channels called “working” channels for allowing the passage of fluids or appliances.

There is therefore a need to provide a medical endoscope provided with at least one sheath or sock, preferably several sheaths, for use in endoscopy, each sheath being easily tearable in order to be taken off for reuse of the endoscope. Advantageously, each sheath is made in a single piece, which ensures good sealing.

There is also a need to have a medical endoscope provided with at least one sheath, and advantageously several sheaths, which is easy to use and which can be reused easily and safely for the patient. There is a need to have a medical endoscope provided with at least one sheath, each sheath can be easily taken off, and allowing the medical endoscope to be reused without requiring putting in place a new sheath, or an additional sterilization step.

### DISCLOSURE OF THE INVENTION

The invention aims to overcome the drawbacks of the state of the art by proposing a medical endoscope provided with at least one sheath or sock. The sheath has an area transparent to the ultrasound and light waves at least in the distal portion, in order to allow satisfactory visualization. The sheath allows protecting the endoscope from any contamination during its insertion into a cavity, a natural duct or a hollow organ. A tearing system makes it possible to easily take off the sheath.

Within the framework of the invention, a “transparent” material or area or element (such as a sheath or a sleeve or a closing portion for example) means that this material or area or element has a transparency index ranging from 40% to 100%. A transparency index can be determined using a UV/visible/near-IR spectrophotometer, by measuring in transmission the emitted light and the light having passed through the material. The transmission index is then determined by the percentage of light having passed through the material compared to the emitted light.

Advantageously, within the framework of the invention, the medical endoscope is provided with several sheaths or socks superimposed on each other. Thus, in the context of the invention, after each use of the medical endoscope, the practitioner can easily take off the outer sheath himself, and immediately reuse or store the medical endoscope for later reuse, without requiring putting in place a new sheath. This is particularly advantageous insofar as this step of putting in place a new sheath can be difficult for the practitioner to perform, in particular in the case where the sheath would include a transparent window then requiring precise adjustment and/or specific equipment to put the sheath in place. The medical endoscope according to the invention is therefore quickly and easily operational again.

Advantageously within the framework of the invention, this stacking of sheaths makes it possible to dispense with the sterilization step after putting in place a new sheath by the practitioner, as is necessary according to the devices of the prior art, since this sterilization is carried out when putting in place the stacked sheaths. Thus, the medical endoscope according to the invention can be reused more safely for the patient because the risks of contamination are more limited. This reuse is also faster and easier for the practitioner who does not necessarily have the equipment necessary to perform sterilization of the sheaths.

Advantageously, within the framework of the invention, thanks to the choice of flexible and stretchable sheaths, the stacking of several sheaths is possible without hindering the movements of the insertion tube, in particular at the level of the bending structure or tip deflection portion, nor causing tearing or damage to the sheaths during these movements of the insertion tube.

More specifically, the medical endoscope according to the invention includes a control handle and an insertion tube equipped with an orientable distal head, the insertion tube being entirely covered with at least one sealed, biocompatible sheath, comprising a sleeve and having a distal end transparent to ultrasound and light waves, the sleeve being flexible, stretchable, matching the shape of the insertion tube and having a hardness of 1 to 70 Shore D and a thickness of 10 μm to 300 μm, characterized in that the sleeve has a tearing system.

The medical endoscope according to the invention advantageously has either of the following characteristics, or a combination thereof:
the tearing system comprises at least one line of least resistance and/or at least one line of greater resistance and/or at least one tearing element;
the lines of least resistance or of greater resistance extend over all or part of the length of the sleeve;
the lines of least resistance are pre-cut lines or lines of least thickness;
the tearing system comprises at least one notch, possibly contiguous to a line of least resistance or of greater resistance, to facilitate the tearing of the sleeve;
the at least one sheath is a single piece, without welding or exogenous supply;
the sleeve is made of a heat-shrinkable elastomeric thermoplastic material;
the heat-shrinkable elastomeric thermoplastic material is selected from polyurethanes, polyamides, polyvinyl chlorides, polytetrafluoroethylenes (PTFE), and polyamide-polyether block copolymers;
the medical endoscope comprises from one to five superimposed sheaths;
the medical endoscope comprises a single sheath.

The invention also relates to a method for preparing an endoscope according to the invention comprising the following steps:
a) providing a medical endoscope including a control handle and an insertion tube equipped with an orientable distal head,
b) disinfecting the medical endoscope,
c) putting in place, by heating, a sheath comprising a sleeve obturated at one of its ends by a closing portion,
d) making a tearing system on the sleeve,
e) if necessary, repeating steps c) and d.

The method for preparing a medical endoscope in accordance with the invention advantageously has either of the following characteristics, or a combination thereof:
the tearing system is made by a cutting member such as knives, punches or serrated blades, by insertion of at least one tearing element, or by coextrusion;
the method comprises a step f) subsequent to step e) consisting in sterilizing the sheath(s);
the method comprises a step of sterilizing the sheath, after step d) and before step e); and
the method comprises a final step of packaging the medical endoscope.

Finally, the invention also relates to a method for preparing a medical endoscope for reuse comprising the following steps:
i) providing a medical endoscope according to the invention provided with an outer sheath already in use,
ii) tearing and taking off the outer sheath to allow the medical endoscope to be reused.

The method for reusing a medical endoscope in accordance with the invention advantageously has either of the following characteristics, or a combination thereof:
in step i), the outer sheath surrounds at least one inner sheath which is used when reusing the endoscope;
the method comprises a step iii) subsequent to step ii), consisting in storing the medical endoscope in a package for reuse.

### BRIEF DESCRIPTION OF THE DRAWINGS

FIG. 1 is a profile view of a medical endoscope whose insertion tube is provided with a sheath in accordance with the invention.

FIG. 2 is a sectional view of an insertion tube provided with a sheath made in a single piece, without welding or exogenous supply.

FIG. 3A is a sectional view of an insertion tube provided with a sheath made by the assembly of a sleeve and of a closing portion in the form of a flat element entirely covering the distal face of the distal head.

FIG. 3B is a sectional view of an insertion tube provided with a sheath made by the assembly of a sleeve and of a closing portion in the form of a flat element having an edge.

FIG. 3C is a sectional view of an insertion tube provided with a sheath made by the assembly of a closing portion and of a sleeve having a return in order to hold the closing portion in position.

FIG. 4 is a sectional view of an insertion tube provided with five superimposed sheaths in accordance with the invention.

FIG. 5 is a plan view of the distal head taken from its distal face.

FIG. 6 is a profile view of a sheath having a tearing system comprising a line of least resistance.

FIG. 7 is a profile view of a sheath having a tearing system comprising a line of least resistance and a notch.

### DESCRIPTION OF THE EMBODIMENTS

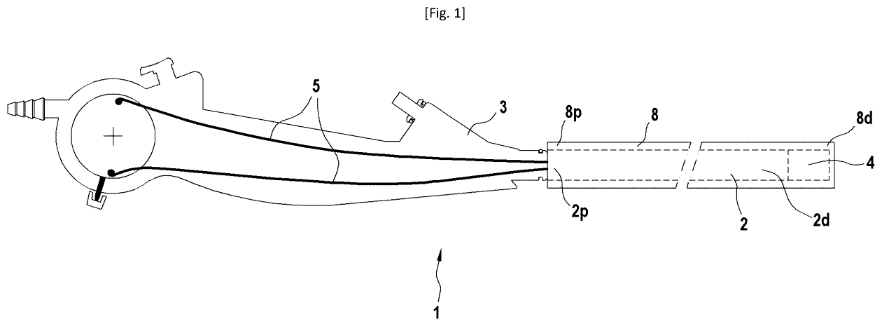

FIG. 1 illustrates a medical endoscope 1 in accordance with the invention, designed to access the interior of a body such as a cavity or a canal for example. Conventionally, a medical endoscope 1 includes an insertion tube 2 having on one side a proximal portion 2p connected to a casing of a control handle 3 and on the opposite side a distal portion 2d which is equipped with a distal head 4. The distal head 4 has a distal face. The distal head is orientable by means of the actuation cables 5. The distal head 4 is equipped with an optical visualization system for illuminating and examining the organ, cavity or duct of the human body inspected. The optical visualization system may be as described in the Application FR 20 00444. In the example illustrated in FIG. 5, the optical visualization system comprises light sources 6 and an optical lens or vision sensor 7. Conventionally, the endoscope 1 also includes an operating or working channel 2c in the insertion tube 2, extending from the control handle 3 to the distal head 4 to allow the supply of various tools and/or fluids and/or the suction of fluids.

Within the framework of the invention, the insertion tube 2 is entirely covered with at least one sheath or sock 8, and preferably from one to five sheaths 8. When the insertion tube 2 is covered with several sheaths 8, these are stacked on top of each other, that is to say they are superimposed on each other. The outermost sheath is referred to as the outer sheath and, when present, the sheath(s) located between the insertion tube and the outer sheath are referred to as inner sheath(s). In the description, “sheath” without mention of “outer” or “inner” designates not only the outer sheath but also the inner sheath(s) when they are present.

Each sheath 8 has a distal end 8d and a proximal end 8p. In order to be used in endoscopy, the distal end 8d of the sheath 8 must be transparent to ultrasound and light waves. Also, the entire sheath 8 or only a portion of it (therefore including at least the distal end 8d) is transparent to ultrasound and light waves. Thus, despite the presence of one or more sheaths, the use of the endoscope is not disrupted, and particularly that of the visualization system.

Within the framework of the invention, each sheath 8 entirely covers the insertion tube 2 by matching its shape, so that each sheath 8 covers the distal face of the distal head 4 by its distal end 8d.

In the context of the invention, each sheath 8 is sealed in order to protect the insertion tube 2 (and possibly one or more inner sheath(s)) from any contamination. In addition, each sheath 8 must be biocompatible in order to be used in endoscopy. Advantageously, each sheath is flexible and stretchable so as not to hinder the movements of the insertion tube, in particular at the level of the bending structure or tip deflection portion.

Each sheath 8 comprises a tubular sleeve 9 obturated at one of its ends by a closing portion 10. This closing portion 10 is either an added element or integrated so that the tubular sleeve 9 and the closing portion 10 form a single piece.

In the context of the invention, the tubular sleeve 9 is flexible and stretchable so as not to disturb the movements of the distal head of the insertion tube 2, and in order to be able to adapt and match the shape of the insertion tube 2 it wraps. The sleeve 9 must be made of biocompatible material in order to be used in endoscopy. The sleeve 9 is also sealed in the context of the invention, in order to protect the insertion tube 2 (and possibly one or more inner sheath(s)) from any contamination. For this, the sleeve 9 has a hardness ranging from 1 to 70 Shore D, and preferably from 20 to 40 Shore D, and a thickness from 10 μm to 300 μm, and preferably from 100 μm to 200 μm. Within the framework of the invention, the resistance is measured with a durometer according to the standard ISO 7619-1:2010, and the thickness is measured using a micrometer.

Preferably, the tubular sleeve 9 is made of an elastomeric thermoplastic material such as for example polyurethanes, polyamides, polyvinyl chlorides, polytetrafluoroethylenes (PTFE), or polyamide-polyether block copolymers. In the context of the invention, the material constituting the tubular sleeve is heat-shrinkable.

The closing portion 10 must be made of a biocompatible material in order to be used in endoscopy. The closing portion 10 is also sealed in the context of the invention, in order to protect the insertion tube 2 (and possibly one or more inner sheaths) from any contamination. The closing portion 10 must also have sufficient resistance properties to prevent any tearing during endoscopy.

The closing portion 10 may be flexible and stretchable to match the shape of the distal head 4, or on the contrary rigid.

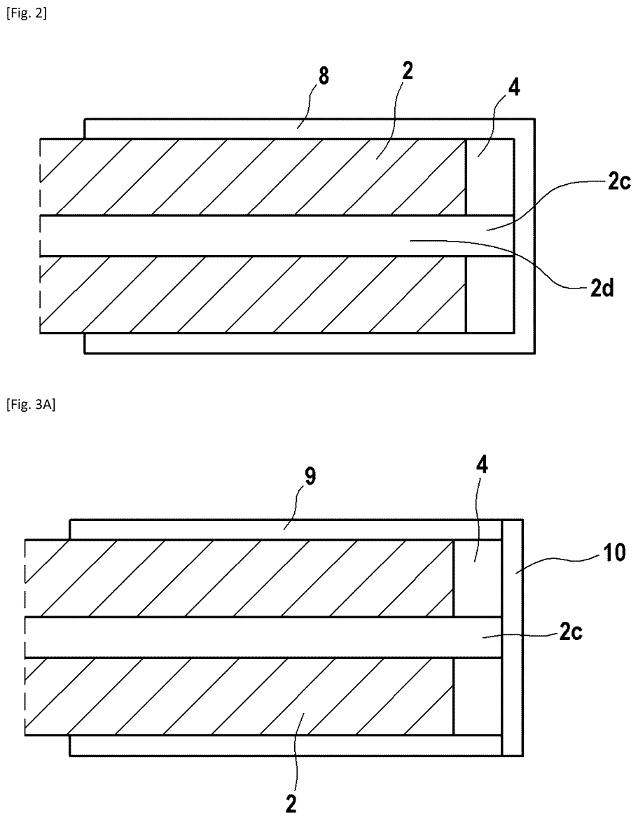

According to a first preferred embodiment of the invention illustrated in FIG. 2, the sheath 8 is made in a single piece, without welding or exogenous supply. According to this embodiment, the sheath 8 is then composed of the sleeve 9 and a closing portion 10 of the same nature. According to this embodiment, the entirety of the sheath 8 is transparent to the ultrasound and light waves. The entirety of the sheath 8 is then made of the same material, which is flexible and stretchable, and has the same hardness. According to this embodiment, the entire sheath 8 may have the same thickness, or on the contrary may have areas of least thickness in order for example to facilitate the tip deflection of the insertion tube 2.

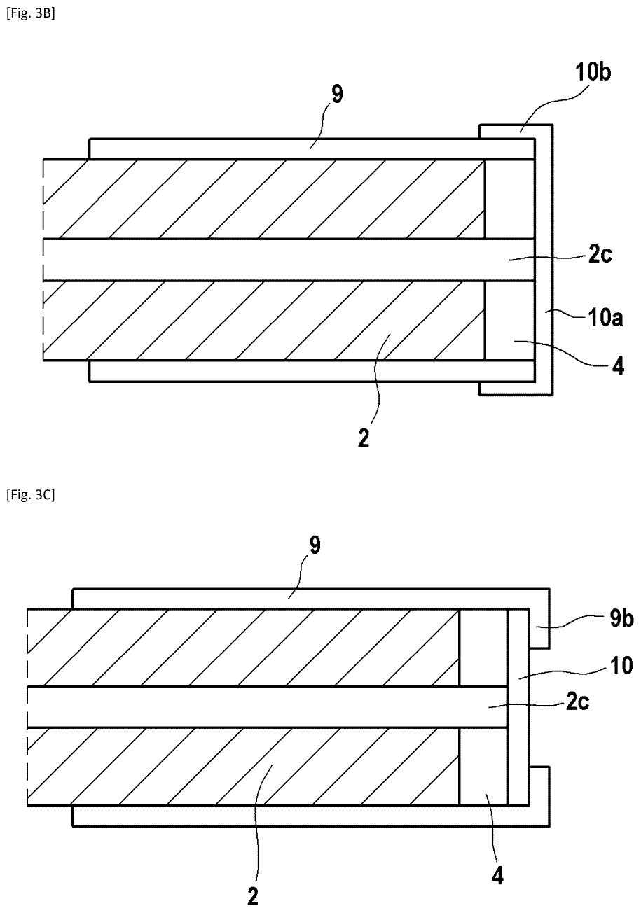

According to a second embodiment illustrated in FIGS. 3A-C, the closing portion 10 and the tubular sleeve 9 are not of the same nature that is to say the sheath 8 is not made in a single piece. The tubular sleeve 9 and the closing portion 10 are then two distinct elements assembled together in a non-removable and sealed manner, for example by welding or by bonding or by heating. In order to ensure the sealing, it is preferable that there is an overlap between the sleeve 9 and the closing portion 10. The sleeve 9 is as described above, and may be transparent to the ultrasound and/or light waves although this is not mandatory. The closing portion 10 is transparent to the ultrasound and light waves, and is placed opposite the optical visualization system. According to this embodiment, the closing portion can be made of glass; polymethyl methacrylate (PMMA); polycarbonate; elastomeric thermoplastic material such as polyurethanes, polyamides, polyvinyl chlorides, polytetrafluoroethylenes (PTFE), or polyamide-polyether block copolymers. According to one particular embodiment, the closing portion 10 and the sleeve 9 are made of the same material.

The closing portion 10 is advantageously attached to the distal face of the distal head 4. Preferably, the closing portion 10 covers the entire distal face of the distal head 4. However, although not preferred, it could be envisaged that the closing portion 10 only covers a portion of the distal face of the distal head 4 including the visualization system. In the latter case, the operating channel 8 is either covered by the closing portion 11 or by the sleeve 10.

As illustrated, the closing portion 10 may be in the form of a flat element with or without edges. Although not illustrated, the closing portion may be in the form of a curved element to adapt to the shape of the distal face of the distal head, it being understood that this element includes a planar area at the level of the visualization system to avoid any optical aberration. In the description of the invention, when mention is made of the flat element, it is understood that a curved element with a planar area intended to be opposite the visualization system could be used instead of this flat element.

According to a first embodiment illustrated in FIG. 3A, the closing portion 10 is in the form of a flat element (without edges), of the same dimensions and same shape as the distal face of the distal head 4. Thus, according to this embodiment, the closing portion 10 and the distal face of the distal head 4 are intended to be placed in a superimposed or coincident position. The sleeve 9 covers the insertion tube 2 up to its distal portion 2d. The sleeve 9 is assembled to the closing portion 10 without overlap between the sleeve 9 and the closing portion 10.

According to a second embodiment illustrated in FIG. 3B, the closing portion 10 is in the form of a flat element 10a extended at right angles by an edge 10b. According to this embodiment, the flat element 10a of the closing portion 10 has the same dimensions and the same shape as the distal face of the distal head 4. Thus, the flat element 10a of the closing portion 10 and the distal face of the distal head 4 are intended to be placed in a superimposed or coincident position. The sleeve 9 covers the insertion tube 2, preferably up to its distal portion 2d. The edge 10b partially covers the sleeve 9, which ensures the sealing of the sheath 8.

According to a third embodiment illustrated in FIG. 3C, the closing portion 10 is in the form of a flat element (without edges), preferably of the same dimensions and the same shape as the distal face of the distal head 4, although it is possible for it to be of smaller dimension than the distal face of the distal head 4. According to this embodiment, the sleeve 9 extends beyond the distal portion 2d of the insertion tube 2 and is extended at right angles by a return 9b which presses the closing portion 10 on the distal head 4.

Each tubular sleeve 9 matches the shape of the insertion tube 2. In other words, the sleeve 9 and the insertion tube are attached (or two sleeves together in the case where the insertion tube 2 is covered with several sheaths), so that no space is present between the sleeve 9 and the insertion tube 2 (or two sleeves together in the case where the insertion tube 2 is covered with several sheaths).

According to a first embodiment illustrated in FIGS. 2 and 3A-C, the insertion tube 2 is covered with a single sheath 8.

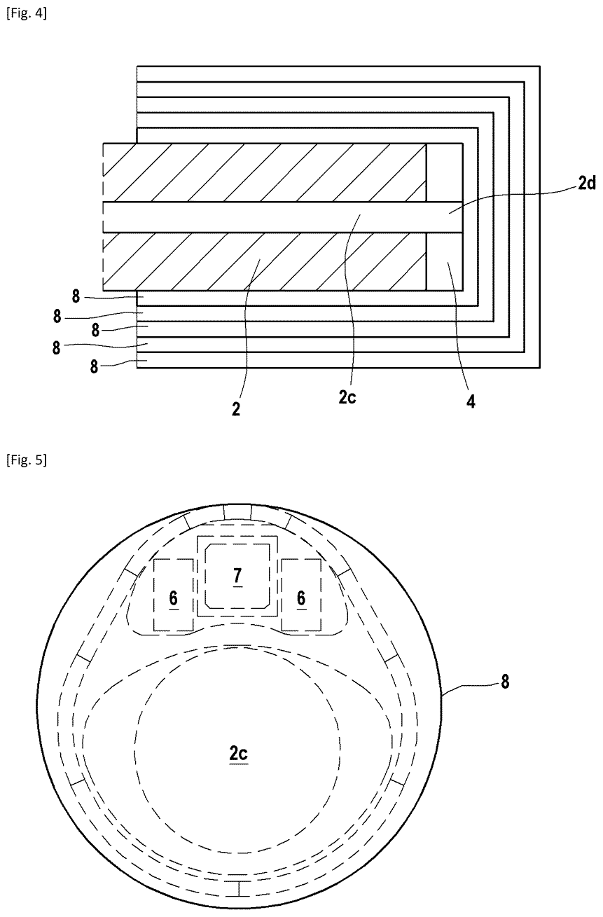

According to a second embodiment, the insertion tube 2 is covered with several sheaths 8, and typically two to five sheaths 8. When several sheaths 8 are present, they are advantageously substantially of the same dimensions and the same shape. According to this embodiment, the different sheaths are superimposed on each other and on the insertion tube 2. In other words, a first sheath 8, called inner sheath, covers the insertion tube 2, then a second sheath 8 covers the first sheath 8, and any subsequent successive sheaths cover the previous sheath. According to the example illustrated in FIG. 4, the insertion tube 2 is covered with five sheaths 8 superimposed on each other: an outer sheath and four inner sheaths. Due to the flexible and stretchable nature of the sleeves, the five sheaths match the shape of the insertion tube 2 without disturbing the movements of the distal head 4.

According to a characteristic of the invention, each sheath 8 has a tearing system 11 to allow removing the sheath 8 after use of the medical endoscope 1. The tearing system extends over the entire length of the sleeve 9, or only over part of the length of the sleeve 9. In all cases, this tearing system 11 must allow the user to at least partially tear the sleeve 9 and partially or, preferably, completely remove the sheath 8 initially present on the insertion tube 2.

The tearing system 11 may consist of one or more lines of least resistance, for example pre-cut lines or lines of least thickness. Alternatively, the tearing system 11 may consist of one or more lines of greater resistance. Alternatively, the tearing system 11 may consist of a tearing element incorporated in the sheath or between two sheaths 8 (or between the sheath 8 and the insertion tube 2), for example a wire of resistance greater than the resistance of the sleeve 9. Alternatively, the tearing system 11 may consist of a notch. According to one particular embodiment, the tearing system 11 consists of:
at least one line of least resistance, for example pre-cut lines or lines of least thickness and/or at least one line of greater resistance, and/or at least one tearing element incorporated in the sheath 8 or placed between two sheaths 8 (or between a sheath 8 and the insertion tube 2) such as for example a wire of resistance greater than the resistance of the sleeve 9,
and at least one notch preferably contiguous to at least one line of least resistance or at least one line of greater resistance.

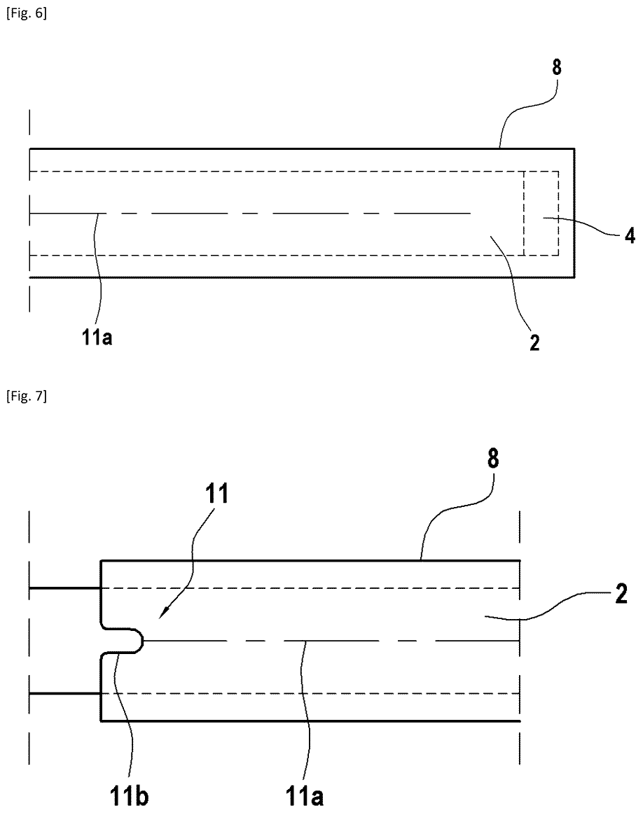

According to the embodiment illustrated in FIG. 6, the tearing system 11 comprises a line of least resistance 11a present over part of the length of the tubular sleeve 9.

According to the embodiment illustrated in FIG. 7, the tearing system 11 comprises a line of least resistance 11a as well as a notch 11b.

FIGS. 2, 3A-C, 6 and 7 illustrate in a simplified manner embodiments of the invention, and these embodiments are not limited to the use of a single sheath. For reasons of simplification, these embodiments have been illustrated when a single sheath is used, but it is clear that these embodiments can also be used when the insertion tube is covered with several sheaths superimposed on each other.

The invention also relates to a method for preparing a medical endoscope 1 entirely covered with at least one sheath 8 as described above. This method comprises the following steps:
a) providing a medical endoscope 1 including a control handle 3 and an insertion tube 2 equipped with an orientable distal head 4,
b) disinfecting the medical endoscope 1,
c) putting in place, by heating, a sheath 8 comprising a sleeve 9 obturated at one of its ends by a closing portion 10,
d) making a tearing system 11 on the sleeve 9,
e) possibly, repeating steps c) and d).

In step b), the disinfection may in particular be carried out by a UV lamp or by cleaning using a disinfectant detergent, for example using wipes impregnated with a disinfectant detergent. During this step, sterilization may also be performed after disinfection. Sterilization of the medical endoscope 1 may, in this case, be carried out for example using ethylene oxide, according to a method well known to those skilled in the art which will not be detailed here.

In step c), the sheath 8 is put in place, by heating, the sleeve 9. More specifically, the insertion tube 2 of the medical endoscope 1 is inserted into the sleeve 9, then the sleeve 9 is heated. Due to the heat-shrinkable nature of the material constituting the sleeve 9, the latter tightens by heating, until it matches the shape of the insertion tube 2.

The method according to the invention may comprise one or more sterilization steps. According to a first embodiment, a sterilization step is carried out after putting each sheath in place, advantageously after step d) of making the tearing system and before step e). According to this first embodiment, sterilization can be carried out using radiation, for example gamma, beta or UV radiation. According to a second embodiment, the method comprises a single sterilization step. According to this second embodiment, the sterilization is carried out only once after putting all the sheaths in place after step e), and allows sterilizing all the sheaths at the same time. According to a second embodiment, the sterilization can be carried out for example by radiation, and in particular gamma radiation. Indeed, this type of sterilization allows sterilizing all the sheaths at the same time.

When the sheath 8 is made in a single piece, without welding or exogenous supply (FIG. 2), then the entire sheath 8 is made of heat-shrinkable material. According to this embodiment, the entire sheath 8 (that is to say the sleeve 9 and the closing portion 10) is heated in step b) in order to attach it to the insertion tube.

When the sheath 8 is composed of a sleeve 9 and a closing portion 10 in the form of a flat element, assembled without overlapping (FIG. 3A), the sleeve 9 is first put in place by heating thanks to its heat-shrinkable nature. Then, the closing portion 10 is assembled to the sleeve 9 by welding or bonding.

When the sheath 8 is composed of a sleeve 9 and a closing portion 10 in the form of a flat element with edges, partially covering the sleeve 9 by its edge 10b (FIG. 3B), the closing portion 10 may be made of heat-shrinkable material. According to this embodiment, the tubular sleeve 9 is first put in place by heating, then the closing portion 10 is put in place and assembled to the tubular sleeve 9 also by heating, or by bonding or welding.

When the sheath 8 is composed of a sleeve 9 extending at right angles by a return 9b which presses the closing portion 10 against the distal face of the distal head 4 (FIG. 3C), the closing portion is first put in place on the distal face of the distal head 4. Then the sleeve 9 is put in place by heating. Due to the heat-shrinkable nature of the material constituting the sleeve 9, the latter matches the insertion tube and allows a sealed assembly with the closing portion 10.

When the tearing system 11 comprises at least one line of least resistance, the latter may be made by a cutting member such as for example knives, punches or serrated blades.

When the tearing system 11 comprises at least one line of greater resistance, the latter may be made for example by coextrusion of two materials of different hardnesses, the first one for making the sheath 8 and the second one, having a hardness greater than that of the sheath, for making the line of greater resistance.

When the tearing system 11 comprises at least one tearing element, the latter may be made by the insertion, into the sheath 8 or between two sheaths 8 (or between the sheath and the insertion tube), of a wire having hardness greater than that of the material constituting the sheath.

When the tearing system 11 comprises at least one notch, the latter may be made by a cutting member such as for example knives, punches or serrated blades.

The method according to the invention may comprise a step f), subsequent to step e), consisting in placing the medical endoscope 1 in sterile packaging in order to protect it from any contamination during its storage and/or transport. A packaging that may be used within the framework of the invention in particular is described in the Application FR 22 09870.

The invention also relates to a method for preparing a medical endoscope 1 comprising the following steps:
i) providing a medical endoscope 1 in accordance with the invention, and provided with an outer sheath already in use,
ii) tearing and taking off the outer sheath to allow the medical endoscope 1 to be reused.

According to a first embodiment, the insertion tube 2 is covered with several sheaths 8. According to this embodiment, the outer sheath surrounds at least one inner sheath which is then used when the medical endoscope 1 is reused.

According to a second embodiment, the insertion tube 2 is covered with a single sheath 8, called outer sheath. In this case, the medical endoscope 1 can be reused only once and therefore does not include a sheath during this reuse.

If necessary, disinfection can be carried out between two uses.

The method according to the invention may comprise a step iii) subsequent to step ii) consisting in placing the medical endoscope 1 in a packaging for reuse. Here again, a packaging that can be used within the framework of the invention is in particular described in the Application FR 22 09870.

1. A medical endoscope including a control handle and an insertion tube equipped with an orientable distal head, the insertion tube being entirely covered with several sheaths superimposed on each other, each sheath being sealed, biocompatible, comprising a sleeve and having a distal end transparent to ultrasound and light waves, each sleeve being flexible, stretchable, matching the shape of the insertion tube and having a hardness of 1 to 70 Shore D and a thickness of 10 μm to 300 μm, characterized in that each sleeve has a tearing system

2. The medical endoscope according to claim 1, wherein the tearing system comprises at least one line of least resistance and/or at least one line of greater resistance and/or at least one tearing element.

3. The medical endoscope according to claim 2, wherein the at least one line of least resistance or the at least one line of greater resistance extend over all or part of the length of the sleeve.

4. The medical endoscope according to claim 2, wherein the at least one line of least resistance are pre-cut lines or lines of least thickness.

5. The medical endoscope according to claim 1, wherein the tearing system comprises at least one notch possibly contiguous to the at least one line of least resistance or the at least one line of greater resistance, to facilitate the tearing of the sleeve.

6. The medical endoscope according to claim 1, wherein each sheath is a single piece, without welding or exogenous supply.

7. The medical endoscope according to claim 1, wherein the sleeve is made of a heat-shrinkable thermoplastic elastomeric material.

8. The medical endoscope according to claim 7, wherein the heat-shrinkable elastomeric thermoplastic material is selected from polyurethanes, polyamides, polyvinyl chlorides, polytetrafluoroethylenes (PTFE), and polyamide-polyether block copolymers.

9. The medical endoscope according to claim 1, comprising from two to five superimposed sheaths.

10. A method for preparing a medical endoscope according to claim 1, comprising the following steps:
a) providing a medical endoscope including a control handle and an insertion tube equipped with an orientable distal head,
b) disinfecting the medical endoscope,
c) putting in place, by heating, a sheath comprising a sleeve obturated at one of its ends by a closing portion,
d) making a tearing system on the sleeve,
e) repeating steps c) and d).

11. The method according to claim 10, comprising a step f) subsequent to step e) consisting in sterilizing the sheath(s).

12. The method according to claim 10, comprising a step of sterilizing the sheath, after step d) and before step e).

13. The method according to claim 10, wherein the tearing system is made by a cutting member such as knives, punches or serrated blades, by insertion of at least one tearing element, or by coextrusion.

14. The method according to claim 10, comprising a final step of packaging the medical endoscope.

15. A method for preparing a medical endoscope for reuse comprising the following steps:
i) providing a medical endoscope according to claim 1 provided with an outer sheath already in use,
ii) tearing and taking off the outer sheath to allow the medical endoscope to be reused.

16. The method according to claim 15, wherein, in step i), the outer sheath surrounds at least one inner sheath which is used when reusing the endoscope.

17. The method according to claim 15 comprising a step iii) subsequent to step ii), consisting in storing the medical endoscope in a package for reuse.
